(19) United States
(12) Patent Application Publication
(10) Pub. No.: US 2023/0159885 A1
(43) Pub. Date: May 25, 2023

(54) METHODS OF INFUSING FOREIGN COMPOUNDS INTO FUNGUS ORGANISMS

(71) Applicant: Blue Sun Mycology Group, LLC, Lebanon, OR (US)

(72) Inventor: Jerred Tudela, Lebanon, OR (US)

(21) Appl. No.: 17/990,494

(22) Filed: Nov. 18, 2022

Related U.S. Application Data
(60) Provisional application No. 63/281,473, filed on Nov. 19, 2021.

(51) Int. Cl. C12N 1/14; A01G 18/00

(52) U.S. Cl. CPC C12N 1/14; A01G 18/00

(57) ABSTRACT

Fungus organism infusion methods including providing a fungus organism; providing a substrate; combining the fungus organism and the substrate; introducing a foreign compound to the substrate to form an infused substrate infused with the foreign compound; maintaining the fungus organism in contact with the infused substrate to promote the fungus organism ingesting the foreign compound from the infused substrate to yield an infused fungus colony; and allowing the infused fungus colony to fruit into a fruit body.

### CROSS REFERENCE TO RELATED APPLICATIONS

This application claims priority to copending U.S. Application Ser. No. 63/281,473, filed on Nov. 19, 2021, which is hereby incorporated by reference for all purposes.

### BACKGROUND

The present disclosure relates generally to methods of producing fungus organisms. In particular, methods of producing fungus organisms incorporating foreign compounds are described.

Fungus organisms are useful in a variety of contexts. Some fungus organisms, such as mushrooms, are a common food source. Other fungus organisms have pharmacological properties used for various therapies, religious ceremonies, and recreational experiences. Mushrooms and other fungus organisms grow on substrates, which provide all the necessary nutrients needed for the mushrooms to form and grow.

As one example of a category of fungus organisms, mushrooms are widely known for their great taste and amazing health benefits. Packed with a ton of essential vitamins and minerals, mushrooms are an excellent addition to one's diet and add flavor to many different recipes. Mushrooms are a low-calorie food that packs a nutritional punch. Loaded with many health-boosting vitamins, minerals, and antioxidants, mushrooms have long been recognized as an important part of any diet. For instance, mushrooms raised with exposure to ultraviolet light are a good source of Vitamin D, an important component in bone and immune health.

Despite the numerous nutritional and pharmacological benefits of fungus organisms, it would be desirable to enhance, expand, and/or customize their nutritional and pharmacological properties. It would be advantageous to infuse fungus organisms with foreign compounds to impart unique properties to the fungus organisms. Further, it would be desirable to form novel compounds with new and beneficial properties within fungus organisms.

Thus, there exists a need for methods of producing fungus organisms with foreign or novel compounds that improve upon and advance the design of known fungus organism production methods. Examples of new and useful methods relevant to the needs existing in the field are discussed below.

Disclosure relevant to fungi production methods is included in U.S. Pat. No. 7,178,285B2, which is incorporated by reference herein for all purposes.

### SUMMARY

The present disclosure is directed to fungus organism infusion methods. The fungus organism infusion methods include various steps. One step involves providing a fungus organism. Another step includes providing a substrate. A further step is directed to combining the fungus organism and the substrate.

An additional step of the methods involves introducing a foreign compound to the substrate to form an infused substrate infused with the foreign compound. A further step includes maintaining the fungus organism in contact with the infused substrate to promote the fungus organism ingesting the foreign compound from the infused substrate to yield an infused fungus colony. An additional step is directed to allowing the infused fungus colony to fruit into a fruit body.

In some examples, maintaining the fungus organism in contact with the infused substrate includes maintaining the fungus organism in contact with the infused substrate for a time period sufficient for the fungus organism to ingest a selected amount of the foreign compound from the infused substrate.

In certain examples, the substrate is in a solid state or a gel state. In such examples, the substrate includes an outer surface and combining the fungus organism and the substrate may include placing the fungus organism on the outer surface of the substrate.

In some examples, the substrate is in a liquid state and defines a liquid substrate. In these examples, combining the fungus organism and the substrate may include dispersing the fungus organism within the liquid substrate.

In select examples, the fungus organism is Psilocybe cubensis, Psilocybe semilanceata, Psilocybe azurescens, Psilocybe mexicana, Psilocybe galindoi, the Golden Halo variant of Psilocybe cubensis, Psilocybe stuntzii, the Enigma variant of Psilocybe cubensis, or the Roller Coaster variant of Psilocybe cubensis. The fungus organism may be a hybrid of two or more fungus organisms.

In certain examples, the fungus organism defines a first fungus organism. In these examples, the method may further include providing a second fungus organism and combining the second fungus organism and the substrate. Certain instances of these method examples include hybridizing the first fungus organism and the second fungus organism.

In some examples, the substrate is manure, wood, or soil. In certain examples, the foreign compound is a metabolite. In select examples, the foreign compound is a phytochemical. In some examples, the foreign compound is a psychoactive compound. The psychoactive compound may be DMT, LSA, LSD, 5-MEO DMT, MDMA, ketamine, or a cannabinoid.

In select examples, the foreign compound defines a first foreign compound. In such examples, the method may include introducing a second foreign compound to the substrate and the infused substrate may be infused with both the first foreign compound and the second foreign compound.

In some examples, introducing the foreign compound to the substrate occurs prior to combining the fungus organism and the substrate.

In certain examples, the foreign compound is in a liquid state. In these examples, the substrate may be in a solid state or a gel state and introducing the foreign compound to the substrate may include spraying the foreign compound onto the substrate and/or onto the fungus organism combined with the substrate.

In some examples, the foreign compound is in a solid state. In such examples, the substrate may be in a solid state or a gel state and introducing the foreign compound to the substrate may include depositing the foreign compound onto the substrate and/or onto the fungus organism combined with the substrate.

In select examples, the substrate is in a solid state or a gel state. In these examples, introducing the foreign compound to the substrate may include impregnating the foreign compound into the substrate.

In some examples, the substrate is in a liquid state and defines a liquid substrate. In such examples, introducing the foreign compound to the substrate may include adding the foreign compound to the liquid substrate to form a solution or a suspension.

In certain examples, the foreign compound is blue popcorn or purple popcorn.

### BRIEF DESCRIPTION OF THE DRAWINGS

FIG. 1 is a flow diagram of a first example of a method of infusing foreign compounds into fungus organisms.

FIG. 2 is a flow diagram of a second example of a method of infusing foreign compounds into fungus organisms.

FIG. 3 is a flow diagram of a third example of a method of infusing foreign compounds into fungus organisms.

FIG. 4 is a flow diagram of a fourth example of a method of infusing foreign compounds into fungus organisms.

FIG. 5 is a flow diagram of a first example of a step for combining a fungus organism and a substrate.

FIG. 6 is a flow diagram of a second example of a step for combining a fungus organism and a substrate.

FIG. 7 is a flow diagram of a first example of a step for introducing the foreign compound onto the substrate and/or onto the fungus organism combined with the substrate.

FIG. 8 is a flow diagram of a second example of a step for introducing the foreign compound onto the substrate and/or onto the fungus organism combined with the substrate.

FIG. 9 is a flow diagram of a third example of a step for introducing the foreign compound onto the substrate and/or onto the fungus organism combined with the substrate.

FIG. 10 is a flow diagram of a fourth example of a step for introducing the foreign compound onto the substrate and/or onto the fungus organism combined with the substrate.

FIG. 11 is a flow diagram of a step for maintaining the fungus organism in contact with the infused substrate to yield an infused fungus colony.

### DETAILED DESCRIPTION

The disclosed methods will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, a variety of method examples are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Spore-producing mushrooms” means any normal, spore producing, physical expression type fruit body belonging to the fungi genus Psilocybe or panaeolus.

“Sporeless mushrooms” means any normal or abnormal physical expression type fruit body belonging to the fungi genera Psilocybe or panaeolus that lacks the ability to produce spores.

“Abnormal mutations” means any fruit body that shows any physical expression not normal to Psilocybe or panaeolus fungi. Such mutations may include, for example, blob type mutations, fin type mutations, coral type mutations, or any fungi with mutated caps or bodies.

“Sclerotia” means the underground growing, truffle type fruit bodies that certain Psilocybe or panaeolus species are capable of producing.

“Monokaryotic” means hyphae and mycelium that contain nuclei of one same genotype. Monokaryotic is interchangeable with heterokaryotic, homokaryotic, and uninucleate.

“Dikaryotic” means mycelium that contain binucleate cells.

“Binucleate” means cells that contain two nuclei.

“Hyphal anastomosis” means cellular fusion between branches of the same or different hyphae or mycelium.

“Hyphae” means individual cellular threads that form when spores germinate.

“Mycelium” means a collection or grouping of hyphae that have formed ropelike threads creating a web-like network.

“Culture” means a product of the cultivation of a living microorganism on a prepared nutrient medium. Such a microorganism may include, for example, Psilocybe or panaeolus mycelium.

“Sub-variant” means a subsidiary variant or subtype of a Psilocybe or panaeolus species. Such subtypes, for example, may include a wild Psilocybe or panaeolus species collected from a certain location or isolated phenotypes of domesticated or wild Psilocybe or panaeolus species.

“Domesticated species” means a Psilocybe or panaeolus species that has been stabilized from generational selection and line breeding.

“Wild species” means a Psilocybe or panaeolus species that has been collected from its natural growing habitat.

“Line breeding” means a form of inbreeding that involves making selections and collecting and growing spores from those selections. The inbreeding process is repeated through multiple generations so that only one or few phenotypes occur more than once within a Psilocybe or panaeolus species sub-variant.

“Vegetatively compatible” means compatible to mate in the vegetative or non-fruiting stage of growth.

### Methods of Infusing Foreign Compounds into Fungus Organisms

With reference to the figures, methods of infusing foreign compounds into fungus organisms will now be described. The reader will appreciate from the figures and description below that the presently disclosed methods advance the state of the art within current fungi production methods.

For example, the novel methods herein enable enhancing, expanding, and customizing the nutritional and pharmacological properties of fungus organisms. Further, the novel methods discussed below infuse fungus organisms with foreign compounds to impart unique properties to the fungus organisms. In some examples, the methods form novel compounds with new and beneficial properties within fungus organisms. The novel compounds can be extracted from fungus organism fruit bodies resulting from the novel methods below for medicinal or commercial use.

### Method Embodiment One

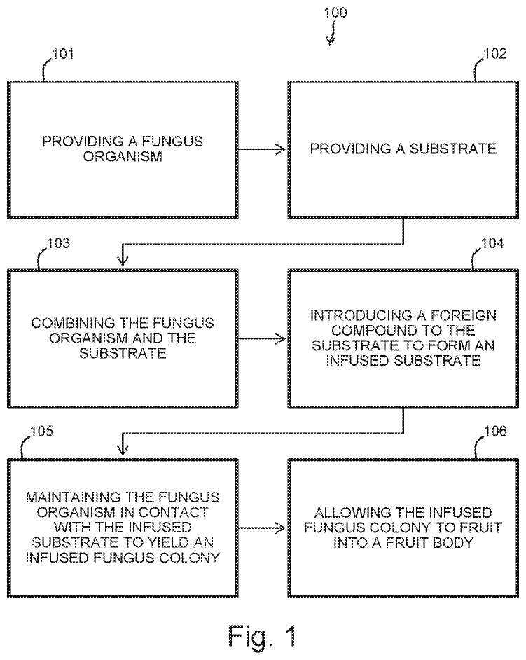

With reference to FIG. 1, a first example of a method of infusing foreign compounds into fungus organisms, method 100, will now be described. Method 100 includes providing a fungus organism at step 101. At step 102, method 100 includes providing a substrate.

Method 100 continues with combining the fungus organism and the substrate at step 103. At step 104, method 100 includes introducing a foreign compound to the substrate to form an infused substrate. Method 100 further includes maintaining the fungus organism in contact with the infused substrate to yield an infused fungus colony at step 105. At step 106, method 100 includes allowing the infused fungus colony to fruit into a fruit body.

In some examples, the method does not include one or more steps of method 100. For example, some method examples start with a fungus organism already on a substrate and thus do not include distinct steps of providing a fungus organism and providing a substrate. In other examples, the method includes additional or alternative steps than described in connection with method 100.

The reader should understand that the numerical step numbers 101 and 102 are simply unique numbers and do not dictate that one step will occur before the other, i.e., that the steps will be carried out in sequential numerical order. For example, introducing a foreign compound to the substrate or fungus organism at step 104 may occur before the fungus organism is transferred to a substrate at step 103.

### Providing a Fungus Organism

The fungus organism provided at step 101 may be any currently known or later developed fungus organism. The fungus organism may be selected from the genus Psilocybe or Panaeolus. Suitable fungus organisms include Psilocybe cubensis, Psilocybe semilanceata, Psilocybe azurescens, Psilocybe mexicana, Psilocybe galindoi, the Golden Halo variant of Psilocybe cubensis, Psilocybe stuntzii, the Enigma variant of Psilocybe cubensis, and the Roller Coaster variant of Psilocybe cubensis. A wide range of fungus organisms are contemplated beyond those expressly described.

In some examples, the fungus organism provided is a hybrid of two or more fungus organisms. Providing a fungus organism at step 101 may include providing a newly developed, hybrid fungus organism. For example, the inventor of this present method has developed novel, hybrid fungus organisms through proprietary methods, and the hybrid fungus organism provided at step 101 may be one created from the inventor's proprietary methods. Additionally or alternatively, the fungus organism provided at step 101 may be a hybrid fungus organism created from other methods of crossbreeding fungi.

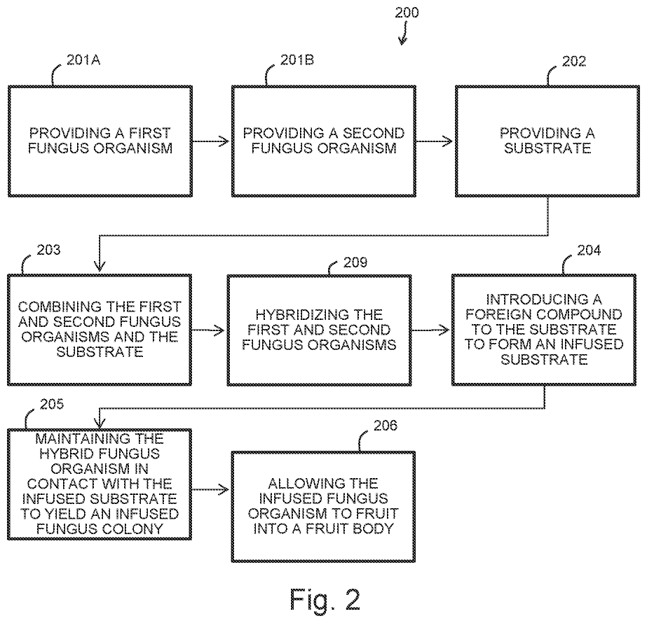

With brief reference to FIG. 2, the reader can see that some methods include providing more than one fungus organism. For example, method 200 shown in FIG. 2 includes providing a first fungus organism at step 201A and providing a second fungus organism at step 201B. The methods disclosed herein may include providing a single fungus organism, two fungus organisms, or multiple fungus organisms.

### Providing a Substrate

The substrate provided at step 102 may be any currently known or later developed substrate. In some examples, the substrate is manure, wood, or soil.

The size, shape, and quantity of the substrate provided at step 102 may be selected to suit the needs of a given application. For example, the substrate may be laid out in trays or beds.

In some examples, the substrate is in a solid state or a gel state. In examples where the substrate is in a solid or gel state, the substrate may include an outer surface.

In certain examples, the substrate is in a liquid state. In examples where the substrate is in a liquid state, the substrate defines a liquid substrate.

### Combining the Fungus Organism and the Substrate

Combining the fungus organism and the substrate at step 103 functions to prepare the fungus organism to be infused with a foreign compound at step 105. Further, combining the fungus organism and the substrate at step 103 enables the fungus organism to replicate into a fungus colony. A further purpose of combining the fungus organism and the substrate at step 103 is to enable the fungus colony (after being infused with a foreign compound at step 105) to fruit into a fruit body at step 106.

Any currently known or later developed method for combining the fungus organism and the substrate may be used for step 103. The methods of combining the fungus organism and the substrate may be manual or automated and scaled to suit any given fungi production objective.

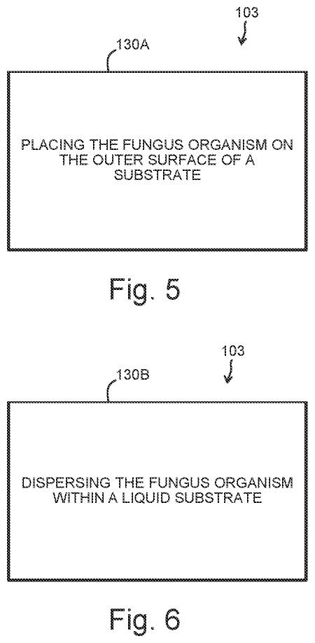

As shown in FIG. 5, in examples where the substrate is in a solid state or a gel state, combining the fungus organism and the substrate at step 103 may include placing the fungus organism on the outer surface of the substrate at step 130A.

In examples where the substrate is in a liquid state, combining the fungus organism and the substrate at step 103 may include dispersing the fungus organism within the liquid substrate at step 130B as shown in FIG. 6.

### Introducing a Foreign Compound

Introducing a foreign compound to the substrate at step 104 facilitates imparting different properties and characteristics to the fungus organism provided at step 101 than it would otherwise have. The different properties and characteristics may enhance or alter the nutritional, medicinal, or recreational value of the fungus organism. Introducing a foreign compound to the substrate at step 104 forms an infused substrate.

The foreign compound introduced at step 104 may be a wide variety of compounds, including metabolites and phytochemicals. In some examples, the foreign compound is a psychoactive compound. The foreign compounds may include organic, semi-synthetic derivatives, or fully synthetic compounds, such as DMT, LSA, LSD, 5-MEO DMT, MDMA, ketamine, cannabinoids, and any non-psychoactive compounds as well. The foreign compound may be any currently known or later developed foreign compound.

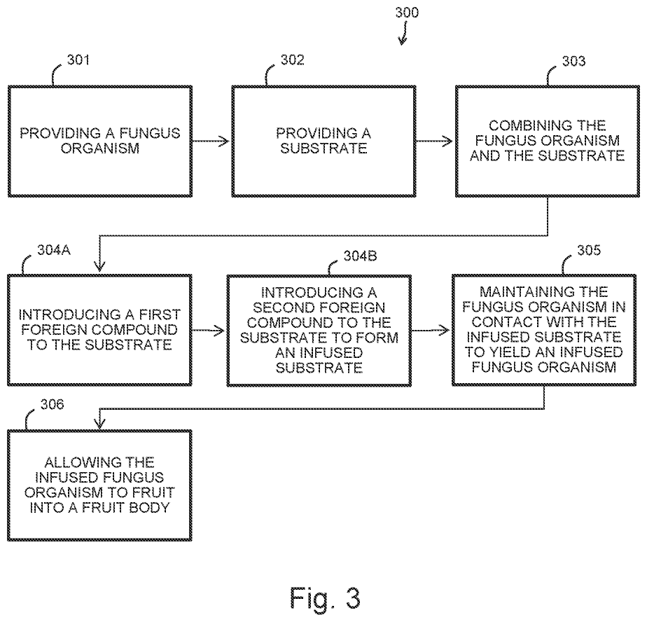

The number of foreign compounds introduced may vary in different examples. In some examples, a single foreign compound is introduced. In other examples, such as shown in FIG. 3, two or more foreign compounds are introduced. Any number of foreign compounds may be introduced, concurrently or sequentially, as desired for a given application.

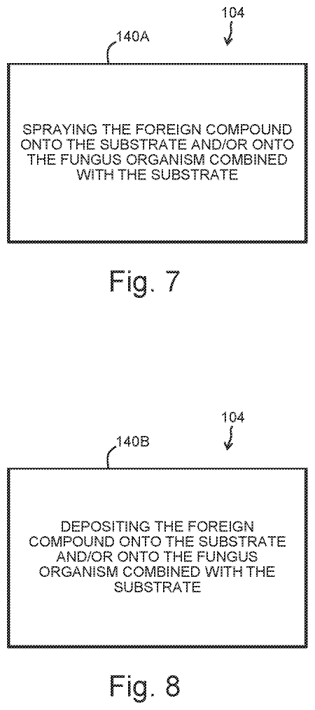

Introducing a foreign compound may be accomplished by any currently known or later developed means. For instance, the foreign compound may be in a liquid state and sprayed on the fungus organism and/or the substrate at step 140A shown in FIG. 7. In certain examples, such as shown in FIG. 8, the foreign compound is in a solid state, such as in a powder or granular form, and is deposited on the fungus colony and/or the substrate at step 140B.

In some examples, such as shown in FIG. 8, the substrate is impregnated with the foreign compound at step 140C to introduce the foreign compound to the substrate at step 104. Additionally or alternatively, a layer of the foreign compound may be applied to the substrate.

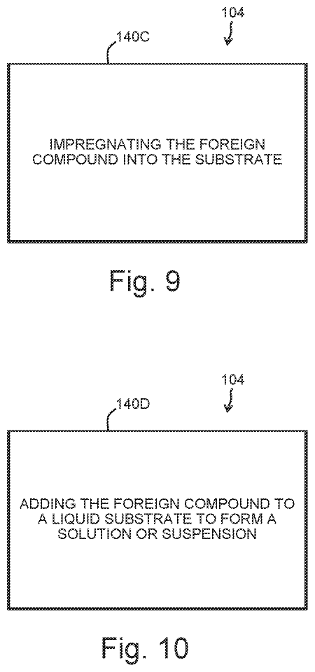

With reference to FIG. 9, the reader can see that in examples where the substrate is liquid, the foreign compound may added to the liquid substrate to form a solution or suspension at step 140D to introduce the foreign compound to the substrate at step 104.

The quantity of the foreign compound introduced at step 104 may be selected over a wide range to meet the needs of a given application. Generally, a higher amount of foreign compound introduced to the substrate and/or fungus organism at step 104 results in higher concentration of the foreign compound in the harvested fungus organism.

In one example, the foreign compound is blue popcorn. In another example, the foreign compound is purple popcorn. Using blue popcorn or purple popcorn as the foreign compound provides the mycelium with new antioxidants that weren't present in the mushroom's chemical composition.

In the blue popcorn and purple popcorn foreign agent examples, the foreign compound also serves as a food source for the fungus organism (also referred to as a fungus colony accounting for organism replicating on the substrate). Providing the fungus colony with a food source allows spores of the fungus colony to germinate. In addition or alternatively to blue or purple popcorn, the food source may be any currently known or later developed nutrient-rich food suitable for a given crossbred fungus organism. Suitable food sources include grains, brans, and mixtures of sawdust and bran.

In certain examples, the foreign compound includes THC or other cannabinoids. The THC or other cannabinoid may be introduced to the fungus colony by adding cannabis to the fungus colony or substrate.

In examples where the foreign compound includes THC or other cannabinoids, the cannabis and substrate may be pressure cooked for a sufficient period of time at a sufficient pressure to activate the cannabinoids before spawning it with the substrate, such as a myceliated grain. In one example, activating the cannabinoids is accomplished by pressure cooking the cannabis and substrate for 40 minutes at 15 PSI, but other times and pressures are also effective. The mushrooms that grow from the resulting activated cannabis infused substrate will be rich in cannabinoids, which weren't previously present in the mushroom's natural chemical composition.

In some examples, the foreign compound includes lysergic acid diethylamide (LSD) or other psychoactive compounds. Suitable psychoactive compounds include DMT, THC, LSD, LSA, and others. The psychoactive compound may be any currently known or later developed psychoactive compound.

The LSD foreign compound may be introduced by spraying a liquid solution of LSD and water on the fungus colony and/or on the substrate. The LSD solution may provide a foreign compound to be incorporated into the fungus organism as well as a source of water that the fungus colony needs to proliferate, survive, and fruit.

### Maintaining the Fungus Organism in Contact With the Infused Substrate

Maintaining the fungus organism in contact with the infused substrate at step 105 functions to infuse the fungus organism with the foreign compound. Infusing the fungus organism with the foreign compound imparts different properties and characteristics to the fungus organism than it would otherwise have. The different properties and characteristics may enhance or alter the nutritional, medicinal, or recreational value of the fungus organism. Maintaining the fungus organism in contact with the infused substrate at step 105 yields an infused fungus colony.

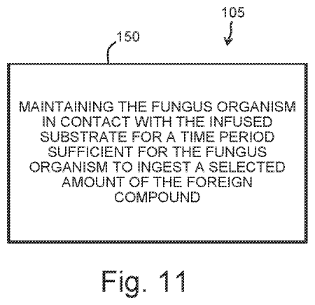

With reference to FIG. 11, the reader can see that maintaining the fungus organism in contact with the infused substrate at step 105 may include a sub-step. For instance, in the example shown in FIG. 11, step 105 includes maintaining the fungus organism in contact with the infused substrate for a selected time period at step 150. The selected time period is a time period sufficient for the fungus organism to ingest or absorb a selected amount of the foreign compound from the infused substrate. The selected amount of the foreign compound may be an amount observed to impart desired properties and characteristics to the fungus organism.

### Allowing the Infused Fungus Colony to Fruit

Allowing the infused fungus colony to fruit at step 106 yields a fruit body. The form and characteristics of the fruit body will be based on the fungus organism and the foreign compound infused in the fungus organism. As a result, the form and characteristics of the fruit body and may be quite vaned.

In some examples, the fruit body is a spore-producing mushroom. In other examples, the fruit body is a sporeless mushroom. In certain examples, the fruit body is a truffle. A wide variety of additional or alternative fruit body phenotypes and characteristics may result from step 106 based on the particular fungus organism used for the fungus colony.

The time required to fruit the infused fungus colony into fruit bodies on the substrate at step 106 will depend on a variety of factors. For example, different fungus organisms will have different fruiting rates and different substrates will enable different fruiting rates for a given fungus organism.

The time, temperature, and pressure conditions for step 106 may be selected within wide ranges as appropriate for given fungus organisms. In some examples, the fungus organism on the substrate is held at room temperature and atmospheric pressure until the fruit bodies occupy a substantial majority of the substrate surface area.

### Additional Embodiments

With reference to the figures not yet discussed, the discussion will now focus on additional method embodiments. The additional embodiments include many similar or identical steps to method 100. Thus, for the sake of brevity, each step of the additional embodiments below will not be redundantly explained. Rather, key distinctions between the additional embodiments and method 100 will be described in detail and the reader should reference the discussion above for steps substantially similar between the different method examples.

### Second Embodiment

Turning attention to FIG. 2, a second example of a fungus organism infusion method, method 200, will now be described. As can be seen in FIG. 2, method 200 includes providing a first fungus organism at step 201A and providing a second fungus organism at step 201B. At step 202, method 200 includes providing a substrate.

Method 200 continues with combining the first and second fungus organisms and the substrate at step 203. At optional step 209, method 200 includes hybridizing the first and second fungus organisms. At step 204, method 200 includes introducing a foreign compound to the substrate to form an infused substrate. Method 200 further includes maintaining the hybrid fungus organism in contact with the infused substrate to yield an infused fungus colony at step 205. At step 206, method 200 includes allowing the infused fungus colony to fruit into a fruit body.

A distinction between method 200 and method 100 is that method 200 provides two distinct fungus organisms at steps 201A and 201B rather than a single fungus organism like described in step 101. Further, method 200 includes combining the first and second fungus organisms with the substrate at step 203 rather than combining a single fungus organism with a substrate like in step 103.

An additional distinction between methods 100 and 200 is that method 200 includes hybridizing the first fungus organism and the second fungus organism at step 209. Step 209 is optional, but enables infusing a hybrid fungus organism with a foreign compound rather than a natural fungus organism. Hybrid fungus organisms with unique and desirable attributes may be created at step 209. The resulting attributes may include enhanced therapeutic capabilities. Infusing a hybrid fungus organism with a foreign compound may yield an infused fungus organism with even greater therapeutic capabilities than the hybrid fungus organism would otherwise have.

### Third Embodiment

Turning attention to FIG. 3, a third example of a fungus organism infusion method, method 300, will now be described. As can be seen in FIG. 3, method 300 includes providing a fungus organism at step 301. At step 302, method 300 includes providing a substrate.

Method 300 continues with combining the fungus organism and the substrate at step 303. As shown in FIG. 3, method 300 includes introducing a first foreign compound to the substrate at step 304A and a introducing a second foreign compound to the substrate at step 304B. Steps 304A and 304B form an infused substrate. Method 300 further includes maintaining the hybrid fungus organism in contact with the infused substrate to yield an infused fungus colony at step 305. At step 306, method 300 includes allowing the infused fungus colony to fruit into a fruit body.

A distinction between method 300 and method 100 is that method 300 introduces two distinct foreign compounds at steps 304A and 304B rather than a single foreign compound like described in step 104. Infusing a fungus organism with two distinct foreign compounds rather than a single foreign compound may enable different or enhanced therapeutic benefits. For example, a fungus organism infused with two foreign compounds may provide two distinct therapeutic benefits deriving from the properties of each foreign compound in a single fruit body vector. Additionally or alternatively, the properties of the foreign compounds may combine to yield a new, distinct, composite therapeutic benefit beyond the therapeutic benefits of either foreign compound alone.

### Fourth Embodiment

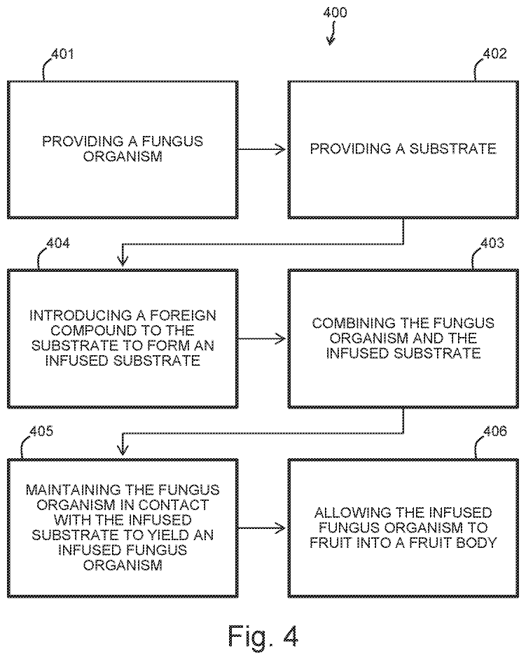

Turning attention to FIG. 4, a fourth example of a fungus organism infusion method, method 400, will now be described. As can be seen in FIG. 4, method 400 includes providing a fungus organism at step 401. At step 402, method 400 includes providing a substrate.

Method 400 continues with introducing a foreign compound to the substrate at step 404. As shown in FIG. 4, method 400 includes combining the fungus organism and the substrate at step 403. Method 400 further includes maintaining the hybrid fungus organism in contact with the infused substrate to yield an infused fungus colony at step 405. At step 406, method 400 includes allowing the infused fungus colony to fruit into a fruit body.

A distinction between method 400 and method 100 is that method 400 introduces a foreign compound to the substrate at step 404 prior to combining the fungus organism and the substrate at step 403. Method 400 demonstrates that the order of these two steps may be reversed. In some cases, infusing the substrate with the foreign compound prior to combining the fungus organism with the substrate is beneficial. In other cases, introducing the foreign compound to the substrate already combined with the fungus organism is beneficial, such as to bring the foreign compound into direct contact with the fungus organism.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

1. A fungus organism infusion method, comprising:
providing a fungus organism;
providing a substrate;
combining the fungus organism and the substrate;
introducing a foreign compound to the substrate to form an infused substrate infused with the foreign compound;
maintaining the fungus organism in contact with the infused substrate to promote the fungus organism ingesting the foreign compound from the infused substrate to yield an infused fungus colony; and
allowing the infused fungus colony to fruit into a fruit body.

2. The method of claim 1, wherein maintaining the fungus organism in contact with the infused substrate includes maintaining the fungus organism in contact with the infused substrate for a time period sufficient for the fungus organism to ingest a selected amount of the foreign compound from the infused substrate.

3. The method of claim 1, wherein:
the substrate is in a solid state or a gel state;
the substrate includes an outer surface; and
combining the fungus organism and the substrate includes placing the fungus organism on the outer surface of the substrate.

4. The method of claim 1, wherein:
the substrate is in a liquid state and defines a liquid substrate; and
combining the fungus organism and the substrate includes dispersing the fungus organism within the liquid substrate.

5. The method of claim 1, wherein the fungus organism is Psilocybe cubensis, Psilocybe semilanceata, Psilocybe azurescens, Psilocybe mexicana, Psilocybe galindoi, the Golden Halo variant of Psilocybe cubensis, Psilocybe stuntzii, the Enigma variant of Psilocybe cubensis, or the Roller Coaster variant of Psilocybe cubensis

6. The method of claim 1, wherein the fungus organism is a hybrid of two or more fungus organisms.

7. The method of claim 1, wherein:
the fungus organism defines a first fungus organism; and
the method further comprises:
providing a second fungus organism; and
combining the second fungus organism and the substrate.

8. The method of claim 7, further comprising hybridizing the first fungus organism and the second fungus organism.

9. The method of claim 1, wherein the substrate is manure, wood, or soil.

10. The method of claim 1, wherein the foreign compound is a metabolite.

11. The method of claim 1, wherein the foreign compound is a phytochemical.

12. The method of claim 1, wherein the foreign compound is a psychoactive compound.

13. The method of claim 12, wherein the psychoactive compound is DMT, LSA, LSD, 5-MEO DMT, MDMA, ketamine, or a cannabinoid.

14. The method of claim 1, wherein:
the foreign compound defines a first foreign compound;
the method further comprises introducing a second foreign compound to the substrate; and
the infused substrate is infused with both the first foreign compound and the second foreign compound.

15. The method of claim 1, wherein introducing the foreign compound to the substrate occurs prior to combining the fungus organism and the substrate.

16. The method of claim 1, wherein:
the foreign compound is in a liquid state;
the substrate is in a solid state or a gel state; and
introducing the foreign compound to the substrate includes spraying the foreign compound onto the substrate and/or onto the fungus organism combined with the substrate.

17. The method of claim 1, wherein:
the foreign compound is in a solid state;
the substrate is in a solid state or a gel state; and
introducing the foreign compound to the substrate includes depositing the foreign compound onto the substrate and/or onto the fungus organism combined with the substrate.

18. The method of claim 1, wherein:
the substrate is in a solid state or a gel state; and
introducing the foreign compound to the substrate includes impregnating the foreign compound into the substrate.

19. The method of claim 1, wherein:
the substrate is in a liquid state and defines a liquid substrate; and
introducing the foreign compound to the substrate includes adding the foreign compound to the liquid substrate to form a solution or a suspension.

20. The method of claim 1, wherein the foreign compound is blue popcorn or purple popcorn.
